(19) United States
(12) Patent Application Publication
(10) Pub. No.: US 2005/0214389 A1
(43) Pub. Date: Sep. 29, 2005

(54) Beta-carotene enriched extract from water hyacinth eichhornia crassipes

(75) Inventors: Vinita V. Panchanadikar, Pune (IN); Swati P. Joshi, Pune (IN); Sundarraj S. Babu, Pune (IN); Sunil R. Bhide, Pune (IN)

Correspondence Address: ERIC A . STEPHENSON;CAMPBELL STEHENSON, 4807 SPICEWOOD SPRINGS ROAD, BUILDING 4 SUITE 201, AUSTIN ,, TX, 78759, US

(73) Assignee: Council of Scientific and Industrial Research, New Delhi (IN)

(21) Appl. No.: 10/811,295

(22) Filed: Mar. 26, 2004

(51) Int. Cl. A61K 35/78; A61K 31/015

(52) U.S. Cl. 424/725; 514/763

(57) ABSTRACT

The present invention provides a convenient and economical process for extraction and separation of β-carotene from water hyacinth Eichhornia crassipes (Pontederiaceae) comprising collecting mature plant of Eichhornia crassipes (5-7 months old, flowering), shade drying, grinding, soaking the powdered plant material in organic solvent to obtain the extract containing carotenoids along with other secondary metabolites which includes some of the value addition components. Enrichment of β-carotene is carried out by dissolving the extract in acetone followed by filtration, and wherein the β-carotene from filtrate is further purified to yield a β-carotene enriched extract.

### FIELD OF THE INVENTION

The present invention provides a process for the extraction of the β-carotene enriched extract from Eichhornia crassipes, commonly known as water hyacinth. More particularly, the present invention provides a process for the extraction of β-carotene enriched extracts wherein the extracts thus obtained were enriched in 9-cis isomers compared to trans isomers.

### BACKGROUND OF THE INVENTION

Water hyacinth (Eichhornia crassipes) has perhaps been the subject of more intense study than any other aquatic plant in recent years. A native of South America, this floating aquatic species has adopted exceedingly well to almost every area into which it has been introduced. Due to its vegetative reproduction and extremely high growth rate, water hyacinth spreads rapidly, clogging drainage, ditches, shading out other aquatic vegetation and also interferes with shipping. Much effort and expense has been devoted to control this prolific weed. Therefore, for the last several years, many investigators have directed their research endeavors to the utilization of this plant species.

Comparison of amino acid content of leaves of water hyacinth with that of grain crop species such as corn, rice, millet, wheat etc. reveals that water hyacinth could make an excellent protein source, with low percentage of ash, and could be used as dietary supplement to balance amino acid intake in a grain diet. Other investigators also showed that comparison is favorable with crude protein and amino acid content of high protein crops such as cottonseeds and soybeans. Therefore, investigators have proposed use of harvested water hyacinth as food supplement both for cattle and humans, as soil additive, as source of energy and fiber. Researchers have demonstrated relationship between nutrient availability and nitrogen and phosphorus content of water hyacinth. The use of water hyacinth as a food supplement appears most promising.

Carotenes are chemical precursors of vitamin A, which is essential to wide variety of physiological processes in animals, including humans. For example, Vitamin A is important in visual sensitivity, and deficiencies of Vitamin A may lead to lack of night vision or even blindness. Vitamin A is also necessary to the proper functioning of epithelial tissues. The carotenes are composed of several forms, including α, β, and γ-carotenes. Of these, the β-carotene isomers have the most vitamin A activity and is the most prevalent in the nature, being found in dark green leafy vegetables, yellow and orange vegetables and fruits and algae. The concentration of β-carotene in edible plants is relatively low and large quantities must be consumed, or else β-carotene must be supplied as a dietary supplement.

To meet growing commercial markets in health and coloring industries, number of methods has been proposed to extract and purify the β-carotene. Few procedures, however, have successfully overcome the considerable obstacles posed by the need to prepare compounds of high purity from natural sources in an economical manner while maintaining acceptability to the consumer and regulatory agencies.

In the prior art the hitherto known processes for extraction of valuable nutrients from water hyacinth and the extraction methods for β-carotene from natural sources are described.

Nagar, P. K.; Saha, Shyamali (Dep. Bot., Calcutta Univ., Calcutta 700 019, India) in ‘Distribution of cytokinin-like activity in different plant parts of the water hyacinth, Eichhornia crassipes’ Physiol. Plant., 64(3), 328-32 (English) 1985, CODEN: PHPLAI. ISSN: 0031-9317. DOCUMENT TYPE: Journal CA Section: 11 (Plant Biochemistry) CA 103:102075 disclose the cytokinin-like activity in extracts of leaf laminae, petioles, shoots, roots, and flowers of young plants of E. crassipes following Sephadex LH-20 column chromatography, using the soybean callus bioassay. In all plant parts, 2 prominent peaks of cytokinin activity, having elution volumes similar to zeatin and zeatin riboside were detected. Putative cytokinin glucoside-like activity was detected only in leaves and flowers. The cytokinin complements of the leaves and the roots were qualitatively different. It would appear that cytokinins supplied by the roots are metabolized in the leaves or certain cytokinins are synthesized in the leaves themselves. The possible significance and distribution of cytokinins in different plant parts in relation to roots is discussed.

Girard, P.; Boillot, M. (Dir. Etud. Rech. Div. Tech. Energ. Nouvelles, EDF, Fr.), in ‘Protein extraction from water hyacinth’, Electr. Fr., Bull. Dir. Etud. et Rech., Ser. A, Nucl., Hydraul., Therm., (3-4), 63-75 (French) 1984. CODEN: EFDNAX. ISSN: 0013-449X. DOCUMENT TYPE: Journal CA Section: 17 (Food and Feed Chemistry) CA 101:22226 disclose a laboratory comparison of 3 external procedures for obtaining a nutritional protein concentrate from water hyacinth, wherein best results were obtained with a Proteinol press. Hypothetical yield from pressing of 100 tons of crude plant material was low (1.42-1.8 ton conc.), with 3-4 times more press cake being obtained. Production of the protein concentrate is not considered profitable.

Lencioni, Livio; Fiorentini, Roberto; Galoppini, Carlo; Brunetti, Nicola (Ist. Ind. Agrar., Univ. Pisa, Italy), in ‘Preparation of leaf protein concentrates from Eichhornia crassipes’, Fitodepur. Impieghi Biomasse Prod., Atti Conv. Int., Meeting Date_{—}1981, 161-9. Edited by: Ghetti, Pier Francesco. Cent. Ric. Prod. Anim.: Reggio Emilia, Italy. (Italian) 1983. CODEN: 50UGAF. DOCUMENT TYPE: Conference CA Section: 17 (Food and Feed Chemistry) CA 100:66875 disclose laboratory and pilot plant experiments wherein water hyacinth (E. crassipes) leaf juice, obtained by several mechanical techniques, was alkalized to pH 8.5 with 2 N NaOH, followed by heating to 85° C., or by treatment with Superfloc A150, to obtain protein concentrates. The protein concentrate obtained by heating contained proteins 40.9, lipids 5.5, fiber 6.7, ash 14.6, and N-free extract 32.3% (dry matter basis). The corresponding values of the concentrate obtained with Superfloc A 150 were 37.7, 6.8, 6.1, 15.4, and 34%.

Anjaneyalu, Yernool V.; Gowda, D. Channe; Neelisiddiah, Belakavadi (Dep. Postgrad. Stud. Res. Chem., Univ. Mysore, Mysore 570 006, India), in ‘Structural features of a polysaccharide from the mucin of water hyacinth’, Phytochemistry, 22(9), 1961-3 (English) 1983. CODEN: PYTCAS. ISSN: 0031-9422. DOCUMENT TYPE: Journal CA Section: 11 (Plant Biochemistry) Section cross-reference(s): 33 CA 100:64940 disclose that the mucin of E. crassipes is a heteropolysaccharide composed of D-xylose, L-galactose, and L-arabinose in the mol ratio 1.3:1.2:1.0. Partial hydrolysis and methylation analysis of the mucin showed the backbone of the polysaccharide to be the trisaccharide repeating unit [(R)4)-D-xylp-(1(R)3)-L-galp-(1(R)2)-L-araf-(1(R)]. All the D-xylopyranosyl residues of the backbone are substituted at O-2, and 1 out of 7 such residues are also substituted at O-3.

Yamamoto, Shigeo; Aoyama, Yoshiko; Kawaguchi, Miho; Iwado, Akimasa; Makita, Masami (Fac. Pharm. Sci., Okayama Univ., Okayama 700, Japan), in ‘Identification and determination of sym-homospermidine in roots of water hyacinth, Eichhornia crassipes Solms’, Chem. Pharm. Bull., 31(9), 3315-18 (English) 1983. CODEN: CPBTAL. ISSN: 0009-2363.DOCUMENT TYPE: Journal CA Section: 11 (Plant Biochemistry) CA 99:209858 identify an unusual polyamine, sym-homospermidine, in water hyacinth (E. crassipes) by gas chromatography-mass spectrometry as its N-ethoxycarbonyl derivative. This polyamine predominated in the root, and its contents, determined by gas chromatography, were in the range of 9.9-46.4 nmol/g fresh wt.

Goswami, P. C.; Nag, B.; Sharma, A. K.; Borthakur, Archana; Singh, H. D.; Baruah, J. N. (Biochem. Div., Reg. Res. Lab., Jorhat 785 006, India), in ‘Water hyacinth as a prospective source of stigmasterol’, Curr. Sci., 52(17), 806-8 (English) 1983. CODEN: CUSCAM. ISSN: 0011-3891. DOCUMENT TYPE: Journal CA Section:. 11 (Plant Biochemistry) Section cross-reference(s): 63 CA 99:191695 disclose the comparatively high content of stigmasterol (0.07% of dry wt.) in water hyacinth (E.crassipes). Roots generally contained less sterol (0.044% of dry mass) than the shoots (0.134), and most of the sterol present was in the free form. Stigmasterol was the predominant sterol; campesterol and β-sitosterol were also found. In the recovery process, stigmasterol could be enriched in the plant mass 10-fold by an anaerobic digestion process.

Lakshminarayana, Gollamudi; Rao, K. Sundar; Pantulu, A. J.; Thyagarajan, G. (Reg. Res. Lab., Hyderabad 500 007, India), in ‘Composition of lipids in roots, stalks, leaves and flowers of Eichhornia crassipes Solms’, Aquat. Bot., 20(3-4), 219-27 (English) 1984. CODEN: AQBODS. ISSN: 0304-3770. DOCUMENT TYPE: Journal CA Section: 11 (Plant Biochemistry) CA 102:109911 disclose that the lipid contents of the roots, leaf stalks, leaves and flowers of E. crassipes (water-hyacinth) were 1.6, 0.9, 14.9, and 5.7%, respectively, on a dry-wt. basis. Non-polar lipids were half the total, whereas glycolipids and phospholipids in approximately equal proportions constituted the remainder, except in leaf stalks, where glycolipids were a larger fraction. Among the non-polar lipids, triglycerols predominated, except for pigments in the leaves. Monogalactosyldiglycerides and digalactosyldiglycerides were the major glycolipids. The main phospholipids were phosphatidylcholine in the roots, phosphatidylglycerol in the leaf stalks and leaves, and phosphatidylethanolamine in the flowers. The major fatty acids were palmitic and linoleic in the roots, linoleic in the leaf stalks, palmitic in the leaves, and linolenic and linoleic in the flowers.

Koizumi, Hideo; Yasui, Akemi; Tsutsumi, Chuichi (Natl. Food Res. Inst., Tsukuba 305, Japan), in ‘Evaluation of inorganic components in water hyacinth, Eichhornia crassipes’, Baiomasu Henkan Keikaku Kenkyu Hokoku, (14), 36-50 (Japanese) 1988. CODEN: BHKHEZ. ISSN: 0913-4549. DOCUMENT TYPE: Journal CA Section: 17 (Food and Feed Chemistry) Section cross-reference(s): 19 CA 114:162759 disclose the cultivation of water hyacinths in river water or in fertilizer solutions. Contents of K, Na, Mg, Ca, Fe, Mn, Zn, Cu, P, and SiO2 in roots, bulbs, and leaves were determined. These plant parts contained a large amount of K, Na, Mg, Ca, and P when water hyacinths were grown in solutions containing high concentrations of these elements. Contents of Fe, Zn, and Cu in bulbs and leaves were much lower than those in roots. Fe, Zn, and Cu were considered to be absorbed by and to accumulate in the roots and to be translocated to the bulbs and leaves to a lesser extent. Absorption of SiO₂ was high; SiO₂ accumulated in the roots, with large amounts being translocated to the bulbs and leaves. The order of the contents of inorganic components in the roots was: K>Ca P>Mg>Fe>Mn>Zn>Cu, and in the bulbs and leaves: K>Ca>P or Mg>Fe or Mn>Zn>Cu, respectively. Plants were divided into juice and residues by pressing. The concentration of K and SiO₂ was high in the juice. K and SiO₂ were removed from plants by pressing and washing; Ca, Mg, Fe, Mn, Zn, and Cu remained in the residues. From the point of utilization of water hyacinth for feed, residues of pressed bulbs and leaves are considered to be desirable.

Ishii, Takeshi; Naohara, Jun (Nishi Nippon Jokaso Kanri Center K. K., Japan). Jpn. Kokai Tokkyo Koho JP 01065101 A2 10 Mar. 1989 Heisei, 3 pp. (Japan) CODEN: JKXXAF CLASS: ICM: C08B037-06. APPLICATION: JP 87-222288 5 Sep. 1987. DOCUMENT TYPE: Patent CA Section: 44 (Industrial Carbohydrates) Section cross-reference(s): 17, 63 CA 110:233520 disclose the preparation of pectin from water hyacinth in high yield by extracting raw or dried water hyacinth with pectin extracting agents. Thus, water hyacinth leaf, water hyacinth leaf stem, water hyacinth root were dried 48 h at 80° C., and 10 g of each dried material was separately extracted with 1 L H₂O at 30° C. for 2 cycles, then extracted with 1 L 0.05 N ammonium oxalate for 1 h at 30° C. for 2 cycles, subsequently extracted with 0.05 N HCl for 1 h at 85° C. for 2 cycles, and finally extracted with 0.05 N NaOH for 1 h at 30° C. for 2 cycles to give pectin with total extracted amount 25.1%, 39.4%, and 10.7%, respectively, vs. 12.5%, 11.7%, 7.6%, respectively using undried water hyacinth.

Yanagisawa, Hiroshi; Kato, Akemi; Hoshiai, Sawa; Kamiya, Akiyoshi; Torii, Naohiro (Biol. Dep., Aichi Univ. Educ., Kariya 448, Japan). Plant Physiol., 85(4), 906-9 (English) 1987, CODEN: PLPHAY. ISSN: 0032-0889. DOCUMENT TYPE: Journal CA Section: 7 (Enzymes) Section cross-reference(s): 11 CA 108:127277 disclose the purification of polyamine oxidase to homogeneity, from leaves of water hyacinth, E. crassipes by the criterion of SDS-PAGE. The enzyme showed a high specificity for spermidine and spermine (Km values of 28 mM and 20 mM, respectively). The optimal pH of the enzyme for both spermidine and spermine was 6.5. The molecular weight of the enzyme established by Sephadex G-200 gel filtration was 87,000, while SDS-PAGE gave a single band at the molecular weight of 60,000. Octamethylenediamine and quinacrine were strong inhibitors of the enzyme, but p-chloromercuribenzoate was without effect. A prosthetic group in the enzyme was identified as FAD.

Zhao, Dajun; Zheng, Shizhang (Fudan Univ., Shanghai 200433, Peop. Rep. China), in ‘Chemotaxis of amino acids in root exudates from Eichhornia crassipes to its rhizospheric Enterobacter sp. F2’, Yingyong Shengtai Xuebao, 7(2), 207-212 (Chinese) 1996. CODEN: YSXUER ISSN: 1001-9332. DOCUMENT TYPE: Journal CA Section: 61 (Water) Section cross-reference(s): 11, CA108:127277 disclose that many types of amino acids are found in root exudates from E.crassipes. F2 can be strongly attracted to amino acids: methionine, gamma amino butyric acid, glycine, alanine, aspartate, serine, valine, and leucine under concentrations of from 10-7 to 10-2 mol/L; some degrees to glutamate, threonine, and histidine; but not to lysine, cysteine, arginine, tyrosine, proline, asparagine, glutamine, isoleucine, phenylalanine, or tryptophan. There exists an optimum chemotaxis concn. range of each possible attractant. The fact that some amino acids can attract the bacteria, but others can not is one of the reasons why E. crassipes and F2 can form a microecosystem.

El-Enany, A. E.; Mazen, A. M. A. (Botany Dep., Faculty Sci., Assiut Univ., Assiut, Egypt), Water, Air, Soil Pollut., 87(1-4), 357-62 (English) 1996. CODEN: WAPLAC. ISSN: 0049-6979, DOCUMENT TYPE: Journal CA Section: 61 (Water) Section cross-reference(s): 11 CA 124:269666 report that water hyacinth plants can accumulate toxic heavy metals and may be useful to elutriate polluted water. A study was conducted to search for the mechanism by which these plants tolerate and accumulate toxic metal ions. Cd was accumulated in the plants against a concentration gradient, mostly as a solution form in the cytoplasm. Isolation and purification of Cd-binding protein with Sephadex A-25 and fractionation on Sephadex G-100 showed that accumulated Cd was associated with 2 major protein fractions. The first fraction, with molecular weight 25-20 kD, contained ˜35% of bound Cd; the second fraction, with molecular weight 12-8 kD, contained ˜40% of bound Cd. The 2 forms were also found in water hyacinth cultivated in Nile River water as a control, although the amount of Cd accumulated was less than those exposed to excess Cd.

Figueiredo, Paulo; Elhabiri, Mourad; Toki, Kenjiro; Saito, Nario; Dangles, Olivier; Brouillard, Raymond (Lab. Chimie Polyphenols, Unvi. Louis Pasteur, Strasbourg 67008, Fr.). Phytochemistry, 41(1), 301-8 (English) 1996; CODEN: PYTCAS. ISSN: 0031-9422. DOCUMENT TYPE: Journal CA Section: 11 (Plant Biochemistry) CA 124:82161 disclose two series of structurally related anthocyanins, extracted from the blue flowers of Evolvulus pilosus and from the blue-purple flowers of E. crassipes, exhibit color stabilities in aqueous solution, at mildly acidic pH. All the pigments possess the same chromophore (delphinidin), but a different pattern of glycosylation and acylation. One of the pigments has an apigenin 7-glucoside molecule (a flavone) attached to the glycosidic chain by two ester bonds with malonic acid, instead of an aromatic acid and is the only known anthocyanin with such a structure. All the molecules studied except one, which has only a 3-gentiobioside as substituent, denote an effect of reduction in the hydration constant, when compared with the parent delphinidin 3-glucoside or 3,5-diglucoside molecules, which supports the existence of intramolecular hydrophobic interactions between the chromophoric skeleton and the acyl or flavonoid groups. The role played by the sugar units on the hydrophobicity/hydrophilicity of the pigments is also discussed.

Yasui, Tadahiko (Fac. Agric., Yamagata Univ., Tsuruoka 997, Japan). Nippon Eiyo, Shokuryo Gakkaishi, 48(5), 391-7 (Japanese) 1995. CODEN: NESGDC. ISSN: 0287-3516. DOCUMENT TYPE: Journal CA Section: 17 (Food and Feed Chemistry) CA 124:54063 disclose a study undertaken to elucidate the extractabilities of true proteins from grasses using new protein extractants, i.e., 0.05 or 0.1 N NaOH soln. contg. 80%, 60%, 40%, 20%, and 0% acetone. The materials for extraction of true proteins were prepared from fresh grasses by treatment with cold acetone. The highest extractability to true proteins for each material was obtained by successive treatment with the above protein extractants. The values obtained were: spinach 84%, white clover 87%, Japanese radish leaf 91%, water hyacinth 96%, bracken 59%. True proteins of spinach and Japanese radish leaf were partly hydrolyzed by these treatments, and hence their practical yields were 60% and 65%, respectively.

Sun, Wenhao; Yu, Ziwen; Guo, Keqin; Yu, Shuwen (Shanghai Inst. Plant Physiol., Acad. Sin., Shanghai 200032, Peop. Rep. China). Zhiwu Shenglixue Tongxun, 27(6), 433-6. (Chinese) 1991. CODEN: CHWSAX. ISSN: 0412-0922. DOCUMENT TYPE: Journal CA Section: 11 (Plant Biochemistry) Section cross-reference(s): 10 CA 116:211137 disclose that an extract of E. crassipes culture inhibited the growth of common algae (Chamydomonas reinhardtii, Chlorella pyrenicidosa, Scenedesmus obliguus, and Anabaena azollae).

Banerjee, Anup; Dubey, Vibha; Banerjee, Keya (Chemistry Department, Sagar University, Sagar 470003, India), J. Oil Technol. Assoc. India (Mumbai, India), 28(4), 107-108 (English) 1996 Oil Technologists' Association of India. CODEN: JOTIAC. ISSN: 0970-4094. DOCUMENT TYPE: Journal CA Section: 9 (Biochemical Methods) CA 128:151388 disclose that lipid analysis in the weed becomes difficult due to the presence of coloring matter which masks the lipids. Moreover, due to the close polarity of phospho- and glycolipids, their separation becomes difficult. In the present study, high performance liquid chromatography (HPLC) has been used for separation of different lipid classes and its advantages over thin layer chromatography (TLC) and thin layer chromatography coupled with flame ionization detector (TLC/FID) are discussed. Two unusual fatty acids from phosphatidylethanolamine and phosphatidylcholine fractions have been characterized.

Foaad, M. A.; Afifi, A. F. Egypt. J. Biotechnol., 7, 101-111, 2000, CA 132:346657 disclose the use of lignocellulosic hydrolyzate of water hyacinth as a sole carbon source for riboflavin production by Aspergillus terreus. The results showed that the highest amount of the riboflavin occurred in the fermentation medium composed of 50% hydrolysate and 50% of basl medium. The composition of the basl medium was as follows (g./l): glucose 10, asparagines 5.98, KH₂PO₄ 2, K₂HPO₄ 2, MgSO₄.7H₂O 1, optimum pH of the medium was at 6.5 and the temperature was 300° C.

Medeiros, Rosalina M. L.: Sabaa Srur, Urmando U. O.: RoquettePinto, Carmen L., Cienc.Tecnol. Aliment., 19(2), 226-230 (Portugese) 1999 CA132:307530 disclose the production of protein concentrate from water hyacinth (E. crassipes) utilized protein extraction with 50 mM sodium hydroxide in a solution/disintegrated plant material ratio of 3:1. Protein preparation used 100 mM HCl at 75-80° C.; the acid solution was added until pH value reached the protein isoelectricity point (pH 3.5). Heating above 80° C. level may denature the proteins. The protein in water hyacinth concentration was of better quality compared to other vegetable protein products, such as alfalfa and soybean flour. The amino acid composition was favorable, especially the presence of tryptophan is valuable since it is commonly low in plant protein products. The presence of all essential amino acids in the concentration suggests its possible use in the production of animal feeds and food supplements for humans.

Ghabbour, Elham A.; Davies, Geoffrey; Lam, Yam-Yuen; Mao, Jingdong; Xing, Baoshan. Prepr. Ext. Abstr. ACS Natl. Meet. Am. Chemical Sco., 39(2), 225-227, 1999 CA 132:21035 disclose the isolation of humic acids (Has) from leaves, stems and roots of water hyacinth and characterized chemically and by IR and NMR spectra. Yields of Has from leaves, stems and roots were 11, 4 and 8% dry wt. respectively.

B. C. Wol Verion and R. C. McDonald, Economic Botany, 32(4), 1978. pp. 363-370 disclose a nutrient analysis of water hyacinth grown in sewage waste waters. Crude protein average 32.9% dry weight in the leaves where it was most concentrated. The amino acid content of water hyacinth leaves was found to compare favorably with that of soybean and cottonseed meal. The vitamin and mineral content of dried water hyacinth exceeded the FAO recommended daily allowance. In many cases it is concluded that in favorable climatic zones water hyacinth grown in enriched medium, such as sewage lagoons, could potentially serve as a substantial dietary supplement or mineral source.

Monsod Jr. Godofredo G. 1981 U.S. Pat. No. 4,251,508 17 Feb. 1981, 3 pp. Cont.-in-part of U.S. Ser. No. 702,612, abandoned 94:153667 disclose that the juice extracted by expeller process from water lily (E.crassipes) leaves contains per 100 g sample vitamin-A (calculated as carotene) 31.64 mg, vitamin-B 0.32 mg, vitamin-B2 1.14 mg, niacin 4.7 mg, protein 23.2 g, chlorophyll 4-8 g, crude fibre 2.3 g, crude lipid 3.2 g, ash 21.5 g, moisture 7.6 g and the balance N-free extract. The dried leaf was shown to contain in 100 g sample thiamine-HCl 0.59 mg, Riboflavin 3.07, vitamin-E 10.60, Pyridoxine-HCl 1.52, vitamin 0.25, niacin 7.94, pantheonic acid 5.56, xanthophyll 48.5, vitamin-B12 1.26, Ca 7.56, Fe 14.3, P 0.93, Mg 849, Zn 2.3, Cu 0.8, Na 1.83, K 3.60 and S 0.45. A useful application of product is the addition of it at a level of 10-15% by weight to human and animal waste as deodorizer.

L. Lareo and R. Bressani Institute of Nutrition of Central America and Panama, Guatemala City, Guatemala U.S. Pat. No. 4,439,629 describe the treatment of algae with calcium hydroxide at an elevated temperature to saponify the chlorophyll and produce a residue, which is then filtered, dried and extracted with a solvent, such as halogenated hydrocarbon or an aliphatic or aromatic hydrocarbon, and recrystallized to yield enriched all-trans β-carotene.

U.S. Pat. No. 5,019,668 to Keat et al., describes the recovery of carotenoids from palm oil by an esterification process using an edible oil and subjecting the mixture to vacuum distillation at a substantially elevated temperature. The concentration of β-carotene in these preparations was descried as being up-to about 1.9%.

U.S. Pat. No. 4,680,314 to Nomura et al describes the process for concentrating algae and extracting β-carotene with an edible oil such as vegetable oil at elevated temperature i.e., 66 to 100° C. The concentration of carotene in the oil extract was reported to be on the order of 1.9%. U.S. Pat. No. 4,713,398, also to Nomura et al., describes composition of carotene prepared from algae at concentration of 0.5% to 7.5% by weight of an edible oil medium.

D. B. Rodriguez, Amaya, Arhivos. LatinoAmericanos De Nutricion, vol.49. No1-S, 1999,pp-38S, teaches that being highly unsaturated, carotenoids are susceptible to isomerization and oxidation during processing and storage of foods. Isomerization of trans-carotenoids to cis-carotenoids, promoted by contact with acids, heat treatment and exposure to light, diminish the colour and the vitamin A activity of carotenoids. The major cause of carotenoid loss, however, is enzymatic and non-enzymatic oxidation, which depends on the availability of oxygen and the carotenoid structure. It is stimulated by light, heat, some metals, enzymes and peroxides and is inhibited by antioxidants. Data on the percentage losses of carotenoids during food processing and storage are somewhat conflicting, but carotenoid degradation is know to increase with the destruction of the food cellular structure, increase of surface area or porosity, length, severity of the processing conditions, storage time and temperature, transmission of light and permeability of oxygen of the packaging. Contrary to lipid oxidation, for which the mechanism is well established, the oxidation of carotenoids is not well understood. It involves initially epoxidation, formation of apocartenoids and hydroxylation. Subsequent fragmentation presumably result in a series of compounds of low molecular masses. Completely losing its colour and biological activities, the carotenoids give rise to volatile compounds, which contribute to the aroma/flavour, desirable in tea and wine and undesirable in dehydrated carrot. Processing can also influence the bioavailability of carotenoids, a topic that is currently of great interest.

James Allen Olson, Arhivos LatinoAmericanos De Nutricion, vol.49. No1-S, 1999,pp-21S teaches that the current knowledge about the bioavailability of provitamin A carotenoid in foods is insufficient, fragmentary and difficult to interpret. Past methods of estimating the vitamin A value of food carotenoids suffer both from uncertainty about the meaning of bioavailability and from the inadequacy of the indicators used in its determination. Reported conversion ratios of β-carotene to vitamin A in humans in vivo, depending on conditions, range from 2:1 to 26:1(μg/μg). Thus, the ratio of 6:1, devised by the World Health Organization, must be considered as a rough average estimate that is not applicable to all diets. Strategies to increase the dietary intake of carotenoid-containing foods should include measures to enhance carotenoid bioavailability.

Adriana Zerlotti Mercandante, Arhivos LatinoAmericanos De Nutricion, vol.49. No1-S, 1999,pp-52S teaches that carotenoids are extremely reactive and consequently unstable due their long system of conjugated double bonds. Several precautions, such as protection against light and oxygen, use of low temperature and antioxidants, analysis in the shortest possible time, should be taken during isolation and chromatography. The food samples, preferably fresh are homogenized and immediately extracted with a suitable organic solvent. Saponification has been employed in order to hydrolyze the carotenoid esters, remove fatty material and destroy chlorophyll. This optional step facilitates subsequent carotenoid separation, identification and quantification. The separation of carotenoids in usually carried out by column chromatography, thin layer chromatography and high performance chromatography, in analytical or preparative scale, on many stationary phases such as silica-gel, alumina, MgO, Ca(OH)₂ and reversed phase material (C₁₈ and C₃₀). The choice of the most suitable chromatographic method depends on the amount of sample, carotenoid composition, resolution, speed and purity required. Examples of carotenoid separation in different stationary phases will be shown and discussed.

Drawbacks of Hitherto Known Processes:

There remains a significant need in the art for a method of producing natural β-carotene composition of high purity, particularly compositions which are enriched in the 9-cis isomer of β-carotene. The method described herein will provide β-carotene in a form, which maintains its anti-oxidant capability, and in a form, which is acceptable to food and health regulatory agencies and to consumers. Moreover, the methods should provide the ability to produce the β-carotene containing composition on a large scale and in an economically feasible manner. The present invention fulfills these and other related aspects.

Water hyacinth a very fast growing weed which is impossible to control by any means. This however is utilized in the present invention in a fruitful manner reducing weed “control” problem. The solid mass of water hyacinth left over after β-carotene extraction, after carbonization, can be utilized for removal of toxic material from effluents of industries like dye and dye intermediate, metallurgical, pharmaceutical, paper and pulp. Other value addition products like stigma sterol also can be obtained as a by-product.

### OBJECTS OF THE INVENTION

The main object of the present invention is to provide an improved, convenient and economically feasible eco-friendly process for the extraction of β-carotene enriched extract from water hyacinth Eichhornia crassipes.

### SUMMARY OF THE INVENTION

Accordingly the present invention provides a process for the extraction of β-carotene from Eichhornia crassipes, the process comprising:
a) drying 5-7 months matured flowering plant material of Eichhornia crassipes to reduce water content;
b) grinding the dried plant material to obtain powdered plant material;
c) soaking the powdered plant material in an organic solvent to obtain a solvent extract;
d) filtering the solvent extract to obtain a filtered extract containing carotenoids and chlorophyll, and residual plant material;
e) re-extracting the residual plant material with an organic solvent to obtain a solvent extract and repeating step (d) to obtain a filtered extract;
f) combining the two filtered extracts of steps (d) and (e) and concentrating the filtered extract under recycling of recovered solvent to obtain a concentrated extract;
g) dissolving the concentrated extract obtained in step (f) in a polar solvent to obtain β-carotene concentrate, removing the polar solvent and separating the β-carotene;

In one embodiment of the invention the Eichhornia crassipes plant material includes entire plant or any of the parts thereof.

In another embodiment of the invention the solvent used in steps (d) and (e) is selected from the group consisting of n-hexane, petroleum ether and chloroform.

In another embodiment of the invention, the drying in step (a) is carried out in shade.

In yet another embodiment of the invention, step (c) is carried out under agitation.

In another embodiment of the invention the polar solvent used in step (g) is selected from the group consisting of methyl ethyl ketone, acetone, methylene dichloride and chloroform.

In another embodiment of the invention the extraction in step (d) and (e) is carried out at ambient temperature and without agitation.

In yet another embodiment of the invention, the drying in step (a) is carried out till the water content in the plant material is reduced by 75 to 80%.

### DETAILED DESCRIPTION OF THE INVENTION

The present invention provides a process for the extraction of β-carotene from Eichhornia crassipes. The process essentially comprises drying preferably in shade, 5-7 months matured flowering plant material of Eichhornia crassipes in order to reduce water content thereof by about 75-80%. The dried plant material is then ground to a powder. The powdered plant material is then soaked in an organic solvent to obtain a solvent extract. This solvent extract is then filtered to obtain a filtered extract containing carotenoids and chlorophyll, and residual plant material. The residual plant material is then subjected to re-extraction using the same organic solvent as above or a different organic solvent and then filtered to obtain a second filtered extract.

The two filtered extracts are combined and then concentrated and the solvent recycled by evaporation. The residual concentrated extract is then dissolved in a polar solvent to obtain a β-carotene rich concentrate, from which the polar solvent is then removed and the β-carotene separated. The entire plant or any part thereof of Eichhornia crassipes can be used as the starting plant material. The organic solvent used for extraction and re-extraction is preferably n-hexane, petroleum ether or chloroform. The soaking of the plant extract is preferably carried out under agitation. In another embodiment of the invention the organic solvent used in step (g) is selected from the group consisting of methyl ethyl ketone, acetone, methylene dichloride and chloroform. The extraction and re-extraction is preferably carried out at ambient temperature and without agitation in order to avoid disrupting the heat sensitive molecule of β-carotene.

It was observed that the yield of β-carotene was observed to be more from aerial parts of the plant material than that from roots. The process of the invention is selective, convenient and economically feasible extraction and separation of β-carotene.

The process of the present invention is described herein below with reference to the examples, which are illustrative and should not be construed to the limit of scope of the present invention in any manner.

### EXAMPLE 1

Plant material Eichhornia crassipes, commonly known as water hyacinth, 5-7 months mature and flowering, was collected from Mulla-Mutha river, Pune, Maharashtra, India. It was allowed to wither for a week at ambient temperature in shade. At the end of one week water content of the plant material was found to be 20-25%. It was powdered and 0.750 kg of the powdered plant material was soaked in petroleum-ether (60-80° C.), 8.0 L, overnight. The extract was filtered, and the residual plant material re-extracted with petroleum ether, 8.0 L as above. From the combined extract, solvent was evaporated to yield extract 7.2 g. The extract (7.2 g) was dissolved in acetone, (50 ml×2). The acetone soluble were filtered from which the solvent was evaporated to yield acetone soluble 5.407 g, free from more polar components. This was subjected for column chromatography using silica gel 100 g(60-120 mesh) acetone: petroleum-ether gradient as eluent and, monitoring the chromatographic separation by TLC to get a fraction, 0.233 g, containing β-carotene. β-carotene content in this fraction was estimated to be 0.01465 g, using HPLC. Percentage of β-carotene was calculated by comparing its area response with that of standard β-carotene. β-carotene obtained from 0.750 kg of dried plant material was found to be 0.001953%. Fraction devoid of β-carotene was utilized for the isolation of other value addition products like stigma sterol or as a component in deodorant formulation.

### EXAMPLE 2

Plant material Eichhornia crassipes, commonly known as water hyacinth, 5-7 months, matured, flowering, was collected from Mulla-Mutha river, Pune, Maharashtra, India. It was allowed to wither for a week at ambient temperature in shade. At the end of one week water content of the plant material was found to be 20-25%. It was powdered and 0.5 kg of the powdered plant material was soaked in petroleum-ether (60-80° C.), 8.0 L, overnight. The extract was filtered, and the residual plant material re-extracted with petroleum ether, 8.0 L as above. From the combined extract solvent was evaporated to yield extract 4.5 g. The extract (4.5 g) was dissolved in acetone, (50 ml×2), the acetone soluble were filtered from which the solvent was evaporated to yield acetone soluble 1.258 g, free from more polar components. This was subjected for column chromatography using silica gel 100 g(60-120 mesh) acetone: petroleum-ether gradient as eluent and, monitoring the chromatographic separation by TLC to get a fraction, 0.455 g, containing β-carotene. β-carotene content in this fraction was estimated to be 0.06836 g using HPLC. Percentage of β-carotene was calculated by comparing its area response with that of standard β-carotene. β-carotene obtained from 0.5 kg of dried plant material was found to be 0.01367%. Fraction devoid of β-carotene was utilized for the isolation of other value addition products like stigma sterol or as a component in deodorant formulation.

### EXAMPLE 3

Plant material Eichhornia crassipes commonly known as water hyacinth, 5-7 months, matured, flowering, was collected from Mulla-Mutha river, Pune, Maharashtra, India. It was allowed to wither for a week at ambient temperature in shade. At the end of one week water content of the plant material was found to be 20-25%. The roots were separated from the aerial parts, powdered and 0.2 kg of the powdered roots were soaked in petroleum-ether (60-80° C.), 2.5 L, overnight. The extract was filtered, and residual plant material was re-extracted with petroleum-ether, 2.5 L as above. From the combined extract solvent was evaporated. The extract 0.4146 g was dissolved in acetone, (25 ml×2), the acetone soluble were filtered from which the solvent was evaporated to yield solubles 0.393 g free from more polar component. This was subjected for column chromatography using silica gel 100 g(60-120 mesh) acetone: petroleum-ether gradient as eluent and monitoring the chromatographic separation by TLC to get a fraction, 0.085 g containing β-carotene. β-carotene content in this fraction was estimated using TLC and HPLC. β-carotene obtained from 0.2 kg dried powdered roots of the plant material was found to be 0.0005455%. Fraction devoid of β-carotene was utilized for the isolation of other value addition products like stigma sterol or as a component in deodorant formulation.

### EXAMPLE 4

Plant material Eichhornia crassipes commonly known as water hyacinth, 5-7 months, matured, flowering, was collected from Mulla-Mutha river, Pune, Maharashtra, India. It was allowed to wither for a week at ambient temperature in shade. At the end of one week water content of the plant material was found to be 20-25%. The aerial parts of the plant were separated from the roots. It was powdered and 0.2 kg of the powdered plant material was soaked in petroleum-ether (60-80° C.), 2.5 L, overnight. The extract was filtered, and residual plant material was re-extracted with petroleum-ether, 2.5 L as above. From the combined extract solvent was evaporated. The extract, 2.0 g was dissolved in acetone, (25 ml×2), the acetone soluble were filtered from which the solvent was evaporated to yield soluble, 0.848 g, free from more polar components. This was subjected for column chromatography using silica gel 100 g (60-120 mesh) acetone:petroleum-ether gradient as eluent and monitoring the chromatographic separation by TLC to get a fraction 0.282 g, containing β-carotene. β-carotene content in this fraction was estimated using HPLC. β-carotene obtained from 0.2 kg of dried aerial parts of the plant material was found to be 0.003100%. Fraction devoid of β-carotene was utilized for isolation of other value added products like stigma sterol or as a component in deodorant formulation.

Advantages of the Invention

1. The present invention describes extraction of β-carotene at ambient temperature without agitation.
2. While extracting β-carotene from the water hyacinth plant, for separation of chlorophyll and other primary and secondary metabolites, saponification was not carried out which saves the process cost as well as process becomes eco-friendly.
3. The solvents used viz. petroleum-ether and acetone can be recovered and recycled thus making the process economically feasible.
4. Residual plant after extraction of β-carotene was carbonized and used for the removal of toxic pollutants from industrial effluents.
5. The fraction devoid of β-carotene may be utilized for obtaining value addition products like stigma sterol and/or as a component in deodorant formulation.
6. As it was clear from prior art that the fast growing water hyacinth couldn't be controlled, the said invention offers promising use of weed thus indirectly helping the weed control.

1. Process for extraction of β-carotene from Eichhornia crassipes, the process comprising:
(a) drying plant material of Eichhornia crassipes to reduce water content;
(b) grinding the dried plant material to obtain powdered plant material;
(c) soaking the powdered plant material in an organic solvent to obtain a solvent extract;
(d) filtering the solvent extract to obtain a filtered extract containing carotenoids and chlorophyll, and residual plant material;
(e) re-extracting the residual plant material with an organic solvent to obtain a solvent extract and repeating step (d) to obtain a filtered extract;
(f) combining the two filtered extracts of steps (d) and (e) and concentrating the filtered extract under recycling of recovered solvent to obtain a concentrated extract;
(g) dissolving the concentrated extract obtained in step (f) in a polar solvent to obtain β-carotene concentrate, removing the polar solvent and separating the β-carotene;

2. Process as claimed in claim 1 wherein the Eichhornia crassipes plant material includes entire plant or any of the parts thereof.

3. Process as claimed in claim 1 wherein the Eichhornia crassipes plant material comprises 5-7 months matured flowering plant material.

4. Process as claimed in claim 1 wherein the solvent used in steps (d) and (e) is selected from the group consisting of n-hexane, petroleum ether and chloroform.

5. Process as claimed in claim 1 wherein the drying in step (a) is carried out in shade.

6. Process as claimed in claim 1 wherein step (c) is carried out under agitation.

7. Process as claimed in claim 1 wherein the polar solvent used in step (g) is selected from the group consisting of methyl ethyl ketone, acetone, methylene dichloride and chloroform.

8. Process as claimed in claim 1 wherein the extraction in step (d) and (e) is carried out at ambient temperature and without agitation.

9. Process as claimed in claim 1 wherein the drying in step (a) is carried out till the water content in the plant material is reduced by 75 to 80%.

10. Process as claimed in claim 1 wherein the β-carotene is separated by column chromatography.
